(19) United States
(12) Patent Application Publication
(10) Pub. No.: US 2026/0261744 A1
(43) Pub. Date: Sep. 3, 2026

(54) CUSTOM DIGITAL CONTENT PRODUCTION AND CONTENT SOURCE SELECTION AND CONFIGURATION METHOD

(71) Applicant: RRC Washington, Inc., Spokane, WA (US)

(72) Inventors: Lawrence BINDER, Spokane, WA (US); Ryan DURGAN, Spokane, WA (US); Andrew M. LEE, Spokane, WA (US)

(21) Appl. No.: 19/163,180

(22) PCT Filed: Mar. 11, 2024
(86) PCT No.: PCT/IB2024/052332

§ 371 Date: Sep. 8, 2025

Related U.S. Application Data
(60) Provisional application No. 63/451,459, filed on Mar. 10, 2023.

(51) Int. Cl. H04N 21/854; H04N 21/239; H04N 21/2743

(52) U.S. Cl. CPC H04N 21/854; H04N 21/2396; H04N 21/2743

(57) ABSTRACT

A computerized method and system for producing digital content from a remote site. A user at a remote site virtually accesses a physical location, wherein an access request from the user is received and, in response to the access request, the user is allowed, or disallowed, virtual access to the physical location. Upon allowance of the virtual access, sources are provided to the user from the physical location, wherein media at the physical location captures storable electronic content for consumption by the user. The user can select from the sources at the physical location in real-time, then configure the curated content into a customized package for deployment.

### CROSS-REFERENCE TO RELATED APPLICATIONS

The present application is a national stage application that claims the benefit of and priority to International Application No. PCT/IB2024/052332 filed on Mar. 11, 2024 which published as WO2024/189516 on Sep. 19, 2024, which claims benefit of U.S. provisional application Ser. No. 63/451,459, filed Mar. 10, 2023, the entire contents of which are incorporated herein by reference.

### FIELD OF THE INVENTION

The present invention is directed to the field of enabling the customizable capture, compilation, configuration, and sharing of multimodal digital content from one or a plurality of sources at a physical location by a user in a remote location.

### BACKGROUND OF THE INVENTION

Current technology enables participants that are physically distant from one another to have some level of presence together in an electronic forum via telephone, over the internet or employing one or a combination or audio-visual means during the conduct of an event at or in relation to a physical location. Current technology also enables the capture of content from participants and other sources at the physical location, or accessing the physical location remotely, or situated proximate to the physical location. Consumers of such content may discreetly access such content if they have appropriate credentials for controlled or confidential content, or if they can otherwise access it from the internet or a streaming source. However, there are no readily available means for participants to have access to a plurality of content materials that are or were captured by various media modes such as audio or video, associated with an event for editing and consumption of the plurality of material content.

There is need then to provide a system that enables a consumer of content to access a plurality of content materials, select from those materials the content they wish to capture, and generate and configure the content into a consumable package that can be accessed and streamed remotely, downloaded onto a personal device, and shared with others electronically.

### SUMMARY OF THE INVENTION

It is the objective to provide a system and method for providing access to storable electronic content in the form of visual, audio, video and other media.

It is further an objective to enable a consumer of content to access a plurality of content materials, select from those materials the content they wish to capture, and generate and configure the content into a consumable package that can be one or more of accessed and streamed remotely, downloaded onto a personal device, and shared with others electronically.

In accordance with various embodiments, the present invention is directed to a system for providing access to storable electronic content in the form of visual, audio, video and other media. The system enables a consumer of content to access a plurality of content materials, select from those materials the content they wish to capture, and generate and configure the content into a consumable package that can be downloaded onto a personal device, and shared with others electronically. The content may include visual, auditory, data augmented/virtual reality, and other on-location or nearby location information.

In some embodiments, the system enables consumers, for example, but not limited to, live or remote participants at an event to have access to a plurality of content materials associated with an event that are or were captured by one of various modes such as audio or video. In some embodiments, the consumers may have no contact with the event.

Accordingly, and more particularly, comprehended is a computerized method, system and medium for producing digital content, comprising the steps of: providing virtual access to a physical location from a remote site, wherein an access request from the user is received and, in response to the access request, the user is allowed, or disallowed, virtual access to the physical location; upon allowance of the virtual access, providing sources to the user from the physical location, wherein media at the physical location captures storable electronic content for consumption by the user; generating an interface to allow the user at the remote site to select from the sources at the physical location in real-time, wherein one or more of the storable electronic content is selected by the user to thereby form curated content; and, recording the curated content into a data file as the user forms the curated content, thereby generating the customized package from the remote site such that the customized package can be deployed. Prior to the step of allowing the user at the remote site to virtually access the physical location, the user is credentialed, which can be pre-arranged. Further, the created data file is a user journey file that includes a time of choice of the curated content such that the user journey file includes a timeline of the sources being selected by the user. As such, the user journey file can be supplemented, edited or played back as part of the deployment. Lastly, in some embodiments, the physical location is a surgical suite, wherein the media is within the surgical suite, thereby forming a virtual surgical suite. Here, the user journey file sourced by the user is a recorded surgery which can be deployed.

Other features and advantages of the present invention will be apparent from the following more detailed description, taken in conjunction with the accompanying drawings which illustrate, by way of example, the principles of the invention.

### BRIEF DESCRIPTION OF THE DRAWINGS

FIG. 1 is a flow chart depicting the overall system and method for remotely producing the digital content.

FIG. 2 is a block diagram further illustrating the integrated, credential-based scalable communication system providing remote access to a physical site.

FIG. 3 is a flow chart depicting how the sources are provided to the user using media from the physical location and the user-selection from the sources.

FIG. 4 is a flow chart depicting the user's configuration of the content and the deployment of the customized package.

Embodiments and the operations shown in these drawings and described in this specification for the instant system and method are computer-implemented systems and methods. This means it can be implemented in digital electronic circuitry, or in computer software, firmware, or hardware, including the modules disclosed in this specification or in combinations of one or more of them. The operations can be implemented as operations performed by a data processing apparatus on data stored on one or more computer-readable storage devices or received from other sources. A data processing apparatus, computer, or computing device may encompass apparatus, devices, and machines for processing data, including by way of example, a programmable processor, a computer, a system on a chip, or multiple ones, or combinations, of the foregoing. The apparatus can include special purpose logic circuitry, for example, a central processing unit (CPU), a graphics processing unit (GPU), a field programmable gate array (FPGA) or an application-specific integrated circuit (ASIC). The apparatus can also include code that creates an execution environment for the computer program in question, for example, code that constitutes processor firmware, a protocol stack, a database management system, an operating system (for example, an operating system or a combination of operating systems), a cross-platform runtime environment, a virtual machine, or a combination of one or more of them. Any reference to “module” means a subsystem or component of the software or methodology which can be integrated or independent and which performs the set of defined functions or tasks. The apparatus and execution environment can realize various different computing model infrastructures, such as web services, distributed computing and grid computing infrastructures. Embodiments can be implemented using computing devices interconnected by any form or medium of wireline or wireless digital data communication (or combination thereof), for example, a communication network. Examples of interconnected devices are a client and a server generally remote from each other that typically interact through a communication network.

Wherever possible, the same reference numbers will be used throughout the drawings to represent the same parts.

### DETAILED DESCRIPTION OF THE INVENTION

### Definitions

As used herein, certain terms will have the meanings as set forth below.

“Computer” broadly means and includes any machine comprising a processor, a memory, and an operating system, capable of interaction with a user or other computer, and includes, without limitation, desktop computers, notebook computers, laptop computers, servers, tablet computers, handheld computers, smart phones, personal digital assistants, hybrids of any of the foregoing (such as Chromebooks), and similar devices that use and/or store data. It should be known a computer program is an executable program of instructions which can be executed on one computer or on multiple computers that are located at one site or distributed across multiple sites and interconnected by a communication network. Generally, a computer will also include, or be operatively coupled to receive data from or transfer data to, or both, one or more mass storage devices for storing data. A computer can be embedded in another device, for example, a mobile device, a personal digital assistant (PDA), a game console, a Global Positioning System (GPS) receiver, or a portable storage device. Devices suitable for storing computer program instructions and data include non-volatile memory, media and memory devices, including, by way of example, semiconductor memory devices, magnetic disks, and magneto-optical disks. The processor and the memory can be supplemented by, or incorporated in, special-purpose logic circuitry. Additionally, processors for execution of a computer program include, by way of example, both general-and special-purpose microprocessors, and any one or more processors of any kind of digital computer. Generally, a processor will receive instructions and data from a read-only memory or a random-access memory or both. The essential elements of a computer are a processor for performing actions in accordance with instructions and one or more memory devices for storing instructions and data.

“Local” pertains to a relationship between two or more computer/electronic devices wherein the devices are physically located in a small geographic area, for example, at the physical location, such as a home, office, or a building, and communicate with each other over a local area network or without using a network, such as by wired or wireless mechanisms. Remote pertains to a relationship that is not local, such as communication over a wide area network, global network, or combinations thereof.

“Control” means use of the system by actuation of electronic devices to control any one or more audiovisual data feeds, visualization and or manipulation of textual and other imagery and data, control of entry and exit from the system, commandeering and physical manipulation of devices such as one or more cameras, laser pointers, lighting, robotic devices, and other instruments and devices that can be controlled by electronic means. The system is contemplated to enable control by one or more of preprogrammed levels of rights within the system based on credentials, manual granting/removal of rights by a user at a physical location and/or by a credentialed or granted remote user.

“Remote server control hub” means a central server (or multiple servers) with installed software used to control and route information (audio, video and data) based on predefined credentials, algorithms and active user decisions.

“Remote site” means a location that is geographically separate from the physical location, the physical location being the local, central or main location, and the “physical location” defined further below.

“Credential” means one or more computer-generated, company-created or user-created variables that are associated with a given user profile that allows the software and/or other users to determine, based on one or a combination of the variables, what access and controls a given user, or group of users, are permitted. In the preferred embodiment herein, the contents of U.S. patent application Ser. No. 18/561,255 are incorporated herein by reference. As such, credentialing occurs prior to or concurrently with access using credential-based scalable communication system with one or more levels of credentialing taught, as disclosed therein and herein.

“In-suite” means and refers to the locus of any one or more of personnel, equipment, electronic devices, and other system elements that are at the physical location.

“User(s)” or “Participant” means and refers to the entities and individuals participating in an electronic meeting, including at least one of each of a physical location and a remote user. In some instances, a remote user is affiliated with an entity, such as a company, and as such, the company is also a participant based on representation by the remote user and optionally the inclusion of logos, branding and other content provided by such participant company.

“Physical location” means and includes any location where any activity is taking place in which a remote user's access may be desirable, apart from the Remote site. In healthcare environments, a physical location may be at a medical care facility, including but not limited to a hospital. In such environments, the term surgical suite broadly includes a room, portion of a room, a suite of multiple rooms, such as but not limited to, an operating room, a procedure room, a dental chair, a catheter lab, a clinic, or a radiology lab.

“Deploy” refers to the process or module of making the customized package available for use after the content has been selected and curated by the remote user, such use available due to the production of the data file, which allows for editing, supplementation (additions), playback, etc. to the user or even shared with a third party such that the third party can partake in the user-experience.

“Consumption” or “consume” refers to the utilization or use of a software package, media, data or information by end-users.

“Real-time or Real time communication” is defined as the near-simultaneous exchange of information over any type of telecommunications service from the sender to the receiver in a connection with negligible or low latency.

“Remote user” means and includes any person or group of persons who are remote from the physical location. In healthcare environments, a remote user may be any one of a drug or medical device company representative, a clinician or any one or group of persons that are affiliated with an allied healthcare entity. In some embodiments, a remote user is a drug or device representative with specialized knowledge, skills and expertise with a device or drug or system used in a surgical suite.

“Augmented reality” or “mixed reality” means an interactive experience of a real-world environment where the objects that reside in the real world are enhanced by computer-generated perceptual information, sometimes across multiple sensory modalities, including visual, auditory, haptic, somatosensory and olfactory.

“Virtual reality” means a computer-generated simulation of a three-dimensional image or environment that can be interacted with in a seemingly real or physical way by a person using special electronic equipment, such as a helmet with a screen inside or gloves fitted with sensors.

“Media” as used herein refers to the various types of textual, image, audio, video, data and interactive type of digital or electronic formats or devices that convey or capture the storable electronic content.

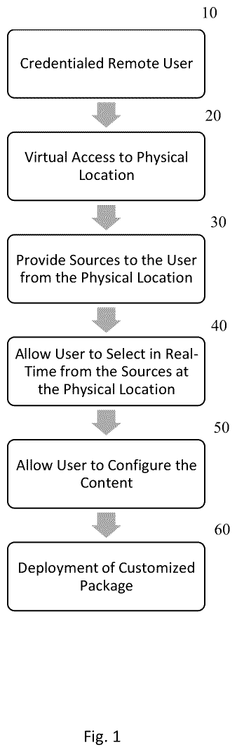

Referencing now FIGS. 1-4, in accordance with various embodiments, the present invention is directed to a system and computerized method for providing access, i.e. virtual access to a physical location 20, to storable electronic content in the form of visual, audio, video and other media by a credentialed remote user 10. Thus, the user is “allowed” such access, consumption, selection, customization and deployment by providing the instant system. The system enables a consumer of content to access a plurality of content materials from sources at the physical location 30, select from those materials the content they wish to capture 40, and generate and configure the content 50 into a consumable package that can be one or more of accessed and streamed remotely, downloaded onto a personal device, and shared with others electronically, i.e. deployed 60. The content may include visual, auditory, data augmented/virtual reality, and other on-location or nearby location information captured from various media, as follows.

An electronic meeting participation system is used, which includes levels of access based on remote user credentials that include at least one and, in some instances, more than two levels of credentialing. In some examples, the system provides for multiple levels of credentialing that include company level credentialing, hospital level credentialing, and in-suite level of credentialing. In an example, company level credentialing includes papers for HIPAA compliance, technical field, device/system specific experience, affiliation with an investigational study, and the like. Hospital level credentialing may include specific requirements of a hospital. And in-suite level credentialing may include affiliation with a particular surgical team or professional or at the discretion of the in-suite director/circulator. There may be additional levels of credentialing that would include access to in-suite controls such as training requirements and the like for use of equipment, such as camera control, robotic controls, use of pointers in the room or onscreen, software control, and three-dimensional room access control.

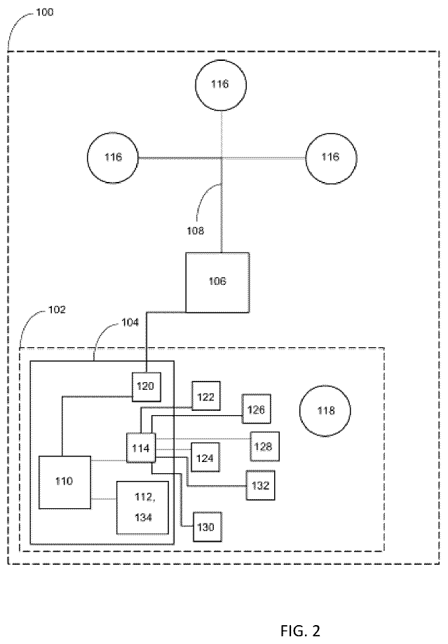

In accordance with the above, first, and with reference particularly to FIG. 2, a remote user 116 at a remote site is allowed to virtually access a physical location, i.e. physical site 102, wherein an access request from the user 116 is received and, in response to the access request, the user 116 is allowed, or disallowed, virtual access to the physical location 102. Prior to the step of allowing the user 116 at the remote site to virtually access the physical location 102, the user is first credentialed. More particularly, the contents of U.S. patent application Ser. No. 18/561,255 are incorporated herein by reference. As such, to allow for credentialed access, provided is an integrated credential-based scalable communication system 100 which provides remote access to a physical site 102. The system 100 includes a dashboard module 104 and a remote server control hub 106 in electronic communication with the dashboard module 104 and an external network 108. The dashboard module 104 includes a computer 110 having a computer processor and computer memory, an electronic display 112 in communication with and driven by the computer 110, dashboard software encoded in the computer memory, and a communication port 114 configured to receive a live stream of audio, video, data, or combinations thereof from the physical site 102. The remote server control hub includes a server processor, server memory, and server software encoded in the server memory. The server software is configured to receive an access request from a remote user 116, assign the remote user 116 a credential having an access profile, and transmit an authorization request to the dashboard module 104. The dashboard software is configured to receive the authorization request and display it for approval on the electronic display 112 for approval by an authorizing agent 118. The server software is configured to receive the live stream from the dashboard module 104, parse the live stream based on the access profile, and, upon approval by the authorizing agent 118, transmit a portion of the live stream corresponding to the access profile to the remote user 116 over the external network 108. The dashboard software, upon commands entered by the authorizing agent 118, is configured to independently modify the access profile, modify the credential, and terminate the live stream. The dashboard module 104 includes an electronic or mechanical switching function 120 that prevents the live stream from being transmitted by the dashboard module 104 to the remote server control hub 106. In some embodiments, as shown in FIG. 2, the integrated credential-based scalable communication system includes a control box 110 that includes at least a physical switch 120 operable to terminate access to all content feed from the physical location.

The live stream originates from the physical site 102. However, additional live streams may be routed through the remote server control hub 106 and may be parsed by the remote server control hub 106 based on credentials or other criteria, which originate from one or more remote users 116. Such additional live streams may be routed from a first remote user 116 to one or more other remote users 116, to the physical site 102, or combinations thereof.

The physical site 102 may be any suitable location, including, but not limited to, a medical treatment suite, a surgical suite, a doctor office, a veterinary suite, or a dental suite. The access profile may include any suitable credential factor, including, but not limited to, HIPAA rules applied to the remote user 116, technical field of the remote user 116, medical tool specific experience of the remote user 116, affiliation of the remote user 116 with an investigational study, hospital credentialing of the remote user 116, affiliation of the remote user 116 with a particular surgical team or professional, or combinations thereof.

The server software being configured to parse the live stream may include selectively blurring or masking portions of a video live stream.

The dashboard software may be configured to transmit an audio live stream to an audio output device 122 located in the physical site 102 which is received from the remote user 116 through the remote server control hub 106 when authorized by the access profile provided by the credential.

The dashboard software may be configured to transmit instructions for remote operation of one or more local devices 124 located in the physical site from the remote user 116 through the remote server control hub 106 when authorized by the access profile provided by the credential. The one or more local devices 124 may include any suitable devices, including, but not limited to, cameras, laser pointers, lighting, robotic devices, surgical instruments, or combinations thereof.

The system may include at least one camera 126 disposed in the physical site 102, the at least one camera 126 being connected to the communication port 114 and providing a video live stream to the dashboard module 104 as at least a portion of the live stream. In one embodiment, wherein the physical site 102 is a surgical suite, the at least one camera 126 is pointed toward at least one of a surgical table, a patient, an x-ray screen, a surgical site of the patient, or an administrator. The at least one camera 126 may include a plurality of cameras 126, and each of the plurality of cameras 126 may be pointed toward a different portion of the surgical suite. The server software may be configured to parse the live stream by restricting access to a camera feed from one or more of the plurality of cameras 126 in addition to or in lieu of selectively blurring or masking portions of a video live stream from any particular camera 126. In one embodiment, the at least one camera 126 has a predetermined field of view with a first portion having a lower sensitivity and a second portion having a higher sensitivity (from a privacy or confidentiality standpoint) according to the access profile of the credential, and the server software is configured to parse the live stream by selectively blurring or masking the second portion of the field of view when the access profile of the credential authorizes access to the first portion but not the second portion.

In addition to or as the electronic display 112, the system 100 may include a monitor 128 which is ceiling-mounted, a wall-mounted, a free-standing, or a projector paired with a screen that is wall-mounted, ceiling-mounted, or free-standing.

The system may further include at least one microphone 130 disposed in the physical site 102 and connected to the communication port 114 to provide an audio live stream to the dashboard module 104 as at least a portion of the live stream.

The server software may be configured to receive a plurality of access requests from a plurality of remote users 116, assign each of the plurality of remote users 116 a user-specific credential having a user-specific access profile, and transmit a user-specific authorization request for each of the plurality of remote users 116 to the dashboard module 104. The server software services a plurality of remote users 116 is configured to receive the live stream from the dashboard module 104, parse the live stream based on each user-specific access profile, and, upon approval by the authorizing agent 118, transmit a portion of the live stream corresponding to the user-specific access profile to the each of the plurality of remote users over the external network 108.

The dashboard module 104 may include an environment scanner 132 which generates a three-dimensional virtual model of the physical site 102 and provides a spatial live stream to the dashboard module 104 as at least a portion of the live stream.

The dashboard software includes an informational module which displays a branding interface 134 on the electronic display 112. The branding interface 134, when activated, pages the remote user 116 through the remote server control hub 106 with an invitation to send an access request to the remote server control hub 106. The branding interface 134 may include a plurality of selection options, and the server software may be configured to page a specific remote user 116 correlated to one of the plurality of selection options amongst a plurality of remote users 116 based on which of the plurality of selection options the authorizing agent 118 selects.

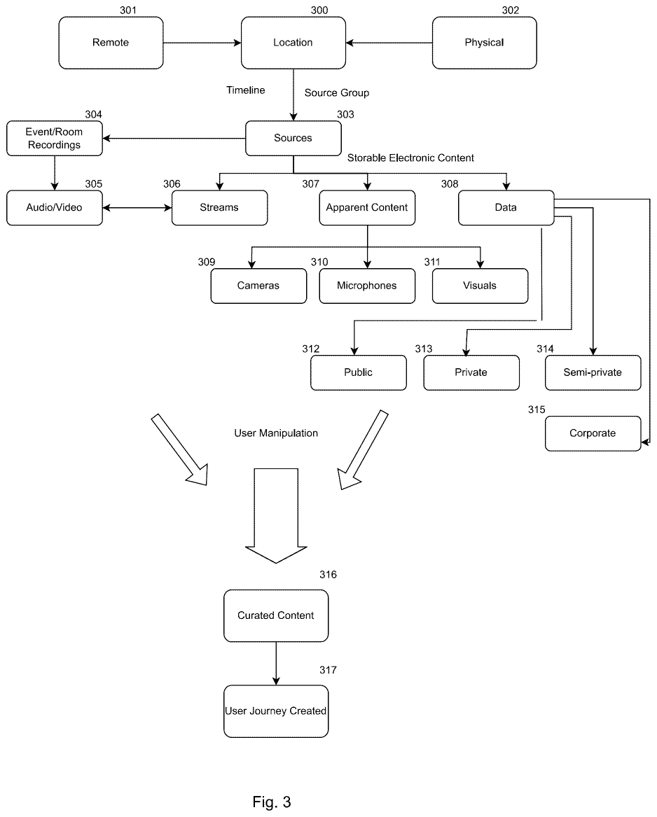

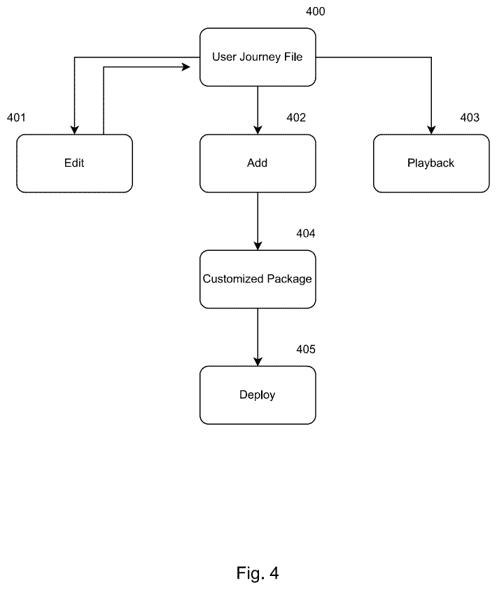

Next, and with reference more particularly to FIGS. 3 and 4, upon allowance of the virtual access 20 to the now-credentialed user 10, sources 303 are provided to the user from the physical location 30, wherein media at the physical location 30 captures storable electronic content from the sources 303 for consumption by the user. This allows the user at the remote site to select from the sources 303 at the physical location 30 in real-time 40, wherein one or more of the storable electronic content is selected by the user to thereby form curated content 316.

The user selects sources through a graphic user interface which allows selection via touch screen, mouse, stylus or other of the visibly displayed sources. Sources may be written in language or number form, icon form or video preview form. One or more sources may be selected simultaneously at any given point in time during the session. As source selections are made, the selections of the user are recorded in a data file specific to that user. The data file will include time of choice and specific choices.

The aggregate of sources 303 for a specific event, film, timeline or subject may be called a source group. Source 303 also pertains to the original, provider(s) of or resource capturing content that is or was or will be curated by any entity. The sources 303 that make up a source group may be prerecorded or live. Sources 303 may be from more than one physical location 302, or from remote locations 301, or from locations proximate to a physical location 302 of interest, any of which is termed herein “location” 300. The storable electronic content, i.e. content, from the sources 303 may include audio and/or video 305 or audio/video-based recordings 304 at a single physical event room 304, such as, for example, a surgical suite, including captured audio and video content 305 from various positions inside and outside the room, or from audio or video streamed content/streams 306 of participants accessing the physical location remotely, or from audio or video streamed content/streams 306 contributed by participants while they are accessing the physical location 302 remotely, or from audio, video, or other content obtained from a location 300 near the physical location 302, such as weather, news or other content, any or all of the foregoing being obtained during the conduct of an event at the physical location 302, or prior to or after the conduct of an event at the physical location 302. By way of non-limiting example, where the physical location 302 is an operating room setting, sources may be apparent content 307 captured using one or more of ceiling wall or floor mounted cameras 309, microphones 310 or other visual sources 311. The sources 303 may also take the form of data 308 captured from encoders, x-ray, c-arm, surgical scope, surgical loops, data from anesthesia and patient monitoring, blood loss, data 308 processed by an AI model, analytics, haptic, body camera, light mounted camera, table or bed mounted camera, instrument mounted camera, contact lens camera, environmental data such as temperature, humidity, particulate, other users, statistics, robotics screens and internal information, 3D scans of physical objects, pictures, MRI data, VR data, neurologic monitoring, audio, and other sources. Note the data 308 may be:
Public data 312. A web-crawler scrubs the internet for geo-located video, pictures and audio to create a Source group based on preset criteria such as time, date, geo-location and range of time +/−.
   Semipublic data 314. Users create or stream sources while opting-in to aggregate their video for other users to experience their perspective from an auditory and visual perspective. This may be related to another product the user is experiencing.
   Private data 313. Users contribute sources to a closed network focused on a particular event, story, film or experience.
   Corporate data 315. A company creates its own private Sources for internal use or for later public consumption.

In general, other potential ways to create sources 303 from a source group, include: (1) a source group specifically created using sources from a movie, television, series or streaming show set; (2) a source group specifically created using sources 303 from a documentary film; (3) a source group specifically created using sources 303 from an animated film; (4) a source group specifically created using sources 303 from a news event; (5) a source group specifically created using sources 303 from a movie where specific information or clues are only available from selecting particular sources 303; (6) a source group specifically created using sources 303 from a sports event; (7) a source group specifically created using sources 303 from a scientific experiment or laboratory; (8) a source group specifically created using sources 303 from, in or on, one or more vehicles or passengers or drivers operating vehicles; (9) a source group specifically created using sources 303 from an event such as a birthday party, wedding, anniversary, holiday party, family gathering, reunion, or the like; (10) a source group specifically created using sources 303 from an event such as an industry trade show or corporate event; (11) a source group specifically created using sources 303 from within a government facility; (12) a source group specifically created using sources 303 from police officers such as body cams and vehicle cams; (13) a source group specifically created using sources 303 from a physical therapy treatment room; (14) a source group specifically created using sources 303 from an exercise training facility or instruction classroom; (15) a source group specifically created using sources 303 from an entire facility or specific areas within a facility such as a warehouse, hospital, prison, manufacturing floor, or the like; (16) a source group specifically created using sources 303 from any event, location over a period of time (for example, a surgical suite, a training room, a seminar room, a gaming theater, a concert, or other venue of interest where or near where content may be captured; (17) a master source group created using multiple source groups as an input.

### Example-Surgical Suite Sources

The invention enables a properly credentialed entity to gain video-based access to a physical location, such as, for example, a surgical suite. The credentialed access includes visual, auditory, data, and other in-suite information access with various levels of control and also provides options for the remote user to commandeer and direct the action of equipment at the physical location. In this example, the instant disclosure addresses elements of the electronic meeting participation system in the context of a surgical suite. Of course, it will be appreciated that this is merely representative, and the invention more broadly relates to an electronic meeting participation system that enables credentialed access and levels of control to one or more remote participants in the context of other physical locations and events, such as but not limited to manufacturing sites, laboratories, etc. Available remote physical controls are interfaced and managed by standard protocol command relays for communicating with specific hardware such as open network video interface forum (ONVIF) and transport layer security (TLS) or other type of standard. The command may be routed through multiple servers and commands may only be available to certain credentialed users. Examples of controls may be to allow remote positioning of cameras or robotics or control of on/off relays present within the remote location.

Therefore, in various embodiments, the system is a surgical suite entry system comprising software programs, front-end user interfaces, backend routing and database access also described as a “software stack”, servers (for capturing, storing and distributing media), audiovisual equipment (cameras and microphones), or combinations thereof, that is used to facilitate a remote live audio and video feed access of the operating room for medical device or drug technical representatives (remote users) during surgical procedures. The surgical suite entry system allows hospital staff to grant or remove individual feed access to a requesting technical representative from inside the operating room.

In this example of access and source 303 selection, a source group is specifically created using sources 303 from within an operating room or surgical procedure room, related to a specific surgical procedure or location. In this manner the physical location is a surgical suite, thus a virtual surgical suite is created for sourcing using the media. Thus, the physical location is a surgical suite and the sources 303 here thereby come from the surgical suite and its various equipment, for example, the surgery for which could also be considered the event 304. This area contemplates two-way interaction. Given proper credentials, the end users can manipulate and operate specific equipment located in the operating room and communicate information to one or more people or objects located in the room.

In some embodiments, the surgical suite entry system includes apparent content 307 captured from a tablet mounted on the wall, or a floor stand. The tablet provides a dashboard for controlling the system. In some embodiments, the surgical suite entry system includes one or more cameras 309 on the ceiling or wall or otherwise affixed in the surgical suite. In some such embodiments, the system includes a combination of cameras 309 on the ceiling and walls. In some embodiments, the surgical suite entry system includes cameras 309 that are one or more of dome, bullet, puck or web style. In some embodiments, at least one camera 309 is pointed at the surgical table (patient). In some embodiments, at least one camera 309 is pointed at an x-ray screen. In some embodiments, at least one camera 309 is pointed at the surgical site (on the patient). In some embodiments, at least one camera 309 is pointed at the circulator/OR administrator. In some embodiments the surgeon is wearing an earpiece to communicate audio 305 directly to the surgeon. In some embodiments, the surgeon is wearing a microphone 310 that allows communication back to the users. In some embodiments, the surgical suite entry system includes a ceiling or wall mounted TV or monitor providing other visuals 311. In some embodiments, the surgical suite entry system includes a projector. In some embodiments, the surgical suite entry system includes external microphones 310 or internal microphones within an electronic device. In some embodiments, the surgical suite entry system is wireless, hardwired or a combination. In various embodiments, the surgical suite entry system includes a data hotspot that is cellular, or satellite-based for data 308 transmission. In some embodiments, the surgical suite entry system allows multiple remote users to access the same live surgical suite audiovisual feed. In some embodiments, the surgical suite entry system allows the technical representative to access the feed of multiple healthcare rooms around the world and specific cameras 309 within those rooms. In various embodiments, the surgical suite entry system allows for remote monitoring of all locations and remote users virtually present in a central software. In some embodiments, the surgical suite entry system allows hospital staff to selectively pause, blur or otherwise distort the transmission of audio, visual or both from the feed to all the remote users from inside the surgical suite. In some such embodiments, the surgical suite entry system allows hospital staff or the remote users to actuate the controls from outside the surgical suite. In some such embodiments, the block function blurs the camera feed and/or the technical representative display instead of blanking it completely. In some embodiments, at least one camera 309 is worn by a person in the room to provide a first-person perspective video feed 305. In some embodiments, at least one camera 309 is mounted on a person in the form of glasses, a piece of headgear or a camera 309 mounted on the body of any person involved in the surgical procedure i.e., surgeon, scrub-tech, anesthesia coordinator, radiology coordinator, nurse, surgical assistant representative, etc. In some embodiments, the surgical suite entry system automatically activates based on credentialing the function to selectively pause, blur, or otherwise distort the transmission of audio, visual or both from the feed to all the remote users from inside the surgical suite. In some embodiments, one or more cameras have the ability to pan tilt or zoom. The actuation of one or more cameras 309 may be under control of a remote user. In some embodiments, the system includes one or more 360-degree cameras that creates one or more 360-degree live video streams of the room. In some such embodiments, at least one remote user is credentialed to view one or more room video feeds from glasses, headset, embedded contact lenses projecting the live video onto the eye or projected video into the remote user's room. In some embodiments, the surgical suite entry system allows the technical representative the ability to change one or all of camera focus locations inside the operating room remotely. In some embodiments, the surgical suite entry system allows the technical representative the ability to remotely manipulate a laser pointer, or other targeted light source, that is physically located within the surgical suite. In some embodiments, the surgical suite entry system allows hospital staff to selectively grant that ability in the surgical suite. In some embodiments, the surgical suite entry system automatically activates remote user remote control based on credentialing. In some embodiments, the surgical suite entry system includes a lidar camera, lidar technology or similar environment scanning technology is used in the room to scan the room in real time for creating a virtual surgical suite, i.e., virtual environment, to be accessed by one or more of the remote users. In some embodiments, the surgical suite entry system includes stitching live video feeds 305 from two or more camera feeds to create a virtual vantage point for the remote user.

Accordingly, here, the operating room sources include but are not limited to the above and one or more of the following: ceiling, wall or floor mounted cameras, microphones, encoders, x-ray, c-arm, surgical scope, surgical loops, data from anesthesia and patient monitoring, blood loss, data processed by an AI model, analytics, haptic, body camera, light mounted camera, table or bed mounted camera, instrument mounted camera, contact lens camera, environmental data such as temperature, humidity, particulate, other users, statistics, robotics screens and internal information, 3D scans of physical objects, pictures, MRI data, VR data, neurologic monitoring, audio, and other surgery-related content. Thus, in summary, the storable electronic content is captured from various media, including but not limited to recordings 304, audio/video 305, streams 306, apparent content 307 and medical data 308.

Now, and with continued reference to FIGS. 3 and 4, after electronic content is captured from the sources 303, the user can manipulate and configure the electronic content into curated content 316. As such a customized package 404 can ultimately be created, thereby generating the customized package 404 from the remote site, which can thereafter be deployed.

As above, the user is allowed to select sources 303 by choosing visual selections (images, words, numbers, icons or other visual elements) on their screen using an input device such as mouse, stylus or touch, in real-time, from a predetermined group of sources 303 from a source group, to create a personalized experience of an event or film. This now personalized experience is called the User Journey, or user journey file 400. Put another way, the system records the user's selection of sources 303 at a particular time along the main timeline of a particular source group, thereby forming a user journey file 400. The user journey file 400 can be added to 402, edited 401 or played back 403. This allows the user to share their selections with other users or play back the sources 303 using the previous source 303 selections. As a non-limiting editing 401 example, users can edit 401 their user journey files 400 to add additional metadata such as notes and transitions between source 303 selections.

The main timeline of a source group starts at the beginning of the earliest source 303 and ends at the end of last source 303. Where sources 303 overlap along the main timeline, they are, in some embodiments, synced, thus can be grouped as a package. Software that facilitates playback of a source group, and its individual sources, allows a viewer/user to experience which source(s) are active while the main timeline of all of the sources 303 progresses simultaneously. This software functions by reading the data file created by a previous user's selections. As described earlier, this file includes exact time and source selections. The current user can invoke this playback of another user's timeline of the same event rather than making their own choices of sources. In turn, they experience the event as the previous user did.

The source group sources 303 all share a common main timeline but have different perspectives and sensory experiences. Moreover, the source group allows the main timeline to be moved forward and backward, paused and restarted. Sources 303 may be available for some or all of the main timeline. Sources 303 may or may not all overlap in time.

The source 303 selections that a user makes are, in some embodiments, recorded in a file that can be shared or recalled later. The information of the selections can be recorded as metadata, a separate digital file or in the user's profile. Users can choose to play back 403 their own User Journey(s) or User Journey(s) created by other users, or start from scratch with a blank User Journey 400, all related to a particular Source group.

While the invention has been described with reference to one or more embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. In addition, all numerical values identified in the detailed description shall be interpreted as though the precise and approximate values are both expressly identified.

What is claimed is:

1. A computerized method for producing digital content, comprising the steps of:
providing virtual access to a physical location from a remote site to a user, wherein an access request from said user is received and, in response to said access request, said user is allowed, or disallowed, said virtual access to said physical location;
upon allowance of said virtual access, providing sources to said user from said physical location from at least some media, said media capturing storable electronic content from said sources for consumption by said user;
generating an interface to allow said user at said remote site to select from said sources at said physical location in real-time, wherein one or more of said storable electronic content is selected by said user to thereby form curated content; and,
recording said curated content into a data file as said user forms said curated content, thereby generating a customized package, wherein said customized package can be further deployed.

2. The method of claim 1, wherein prior to the step of providing said virtual access to said physical location, said user is credentialed.

3. The method of claim 1, wherein said media can further be controlled by said user using standard protocol command relays including commands communicating with said media.

4. The method of claim 1, wherein said data file is a user journey file, said user journey file including a time of choice of said curated content by said user such that said user journey file includes a timeline of said sources being selected by said user.

5. The method of claim 4, wherein said user journey file can be supplemented, edited or played back by either said user or a third party such that said third party can experience said customized package generated by said user.

6. The method of claim 5, wherein said physical location is a surgical suite, wherein said media is within said surgical suite, thereby forming a virtual surgical suite; and, wherein said user journey file sourced by said user is a recorded surgery available for deployment.

7. A non-transitory computer-readable medium with stored instructions, said instructions when executed by a processor, cause said processor to:
provide virtual access to a physical location from a remote site to a user, wherein an access request from said user is received and, in response to said access request, said user is allowed, or disallowed, virtual access to said physical location;
upon allowance of said virtual access, provide sources to said user from said physical location from at least some media, said media capturing storable electronic content from said sources for consumption by said user;
generate an interface to allow said user at said remote site to select from said sources at said physical location in real-time, wherein one or more of said storable electronic content is selected by said user to thereby form curated content; and,
record said curated content into a data file as said user forms said curated content, thereby generating a customized package, wherein said customized package can be further deployed.

8. The computer-readable medium of claim 7, further comprising instructions that when executed causes said processor to credential said user.

9. The computer-readable medium of claim 7, wherein said media can further be controlled by said user using standard protocol command relays including commands communicating with said media.

10. The computer-readable medium of claim 7, wherein said data file is a user journey file, said user journey file including a time of choice of said curated content by said user such that said user journey file includes a timeline of said sources being selected by said user.

11. The computer-readable medium of claim 10, wherein said user journey file can be supplemented, edited or played back by said user or a third party such that said third party can experience said customized package generated by said user.

12. The computer-readable medium of claim 11, wherein said physical location is a surgical suite, wherein said media is within said surgical suite, thereby forming a virtual surgical suite; and, wherein said user journey file sourced by said user is a recorded surgery available for deployment.

13. A system for providing remote access to a physical site, said system comprising:
a dashboard module including:
a computer having a computer processor and computer memory;
dashboard software encoded in said computer memory; and
a communication port configured to receive a live stream of audio, video, data, or combinations thereof, said live stream originating from said physical site;
a remote server control hub in electronic communication with said dashboard module and an external network, said remote server control hub including:
a server processor;
server memory; and
server software encoded in said server memory, said server software including stored instructions, said instructions when executed by said server processor, causes said processor to:
provide virtual access to a physical location from a remote site to a user, wherein an access request from said user is received and, in response to said access request, said user is allowed, or disallowed, virtual access to said physical location;
upon allowance of said virtual access, provide sources to said user from said physical location from at least some media, said media capturing storable electronic content from said sources for consumption by said user;
generate an interface to allow said user at said remote site to select from said sources at said physical location in real-time, wherein one or more of said storable electronic content is selected by said user to thereby form curated content; and,
record said curated content into a data file as said user forms said curated content, thereby generating a customized package, wherein said customized package can be further deployed.

14. The system of claim 13, further comprising instructions that when executed causes said processor to credential said user.

15. The system of claim 13, wherein said media can further be controlled by said user using standard protocol command relays including commands communicating with said media.

16. The system of claim 13, wherein said data file is a user journey file, said user journey file including a time of choice of said curated content by said user such that said user journey file includes a timeline of said sources being selected by said user.

17. The system of claim 16, wherein said user journey file can be supplemented, edited or played back by said user or a third party such that said third party can experience said customized package generated by said user.

18. The system of claim 17, wherein said physical location is a surgical suite, wherein said media is within said surgical suite, thereby forming a virtual surgical suite; and, wherein said user journey file sourced by said user is a recorded surgery available for deployment.
